(19) United States
(12) Patent Application Publication
(10) Pub. No.: US 2024/0393251 A1
(43) Pub. Date: Nov. 28, 2024

(54) COMBINED RAMAN/SINGLE MOLECULE JUNCTION SYSTEM FOR CHEMICAL AND BIOLOGICAL ANALYSIS

(71) Applicant: THE REGENTS OF THE UNIVERSITY OF CALIFORNIA, Oakland, CA (US)

(72) Inventor: Joshua Hihath, Woodland, CA (US)

(73) Assignee: THE REGENTS OF THE UNIVERSITY OF CALIFORNIA, Oakland, CA (US)

(21) Appl. No.: 18/738,277

(22) Filed: Jun. 10, 2024

Related U.S. Application Data
(63) Continuation of application No. PCT/US2022/081782, filed on Dec. 16, 2022.
(60) Provisional application No. 63/290,803, filed on Dec. 17, 2021.

(51) Int. Cl. G01N 21/65; G01N 27/403; G01N 33/487; G16B 40/30

(52) U.S. Cl. CPC G01N 21/658; G01N 27/403; G01N 33/48721; G16B 40/30

(57) ABSTRACT

An apparatus, system and methods that allows simultaneous acquisition of tunneling currents and Raman spectra for molecular sensing, detection, identification or sequencing. The apparatus has a substrate with a surface channel or transverse bore for orienting a target between electrodes of a single molecule break junction. A Raman spectrometer, with excitation source and detector, is configured to simultaneously interrogate the target at the junction. A data acquisition unit receives spectral data from the Raman spectrometer detector and electrical data from the electrodes individually or simultaneously. The acquired data is recorded and analyzed used to identify features of the target and the identification may be assisted by machine learning algorithms.

### CROSS-REFERENCE TO RELATED APPLICATIONS

This application claims priority to, and is a 35 U.S.C. § 111 (a) continuation of, PCT international application number PCT/US2022/081782 filed on Dec. 16, 2022, incorporated herein by reference in its entirety, which claims priority to, and the benefit of, U.S. provisional patent application Ser. No. 63/290,803 filed on Dec. 17, 2021, incorporated herein by reference in its entirety. Priority is claimed to each of the foregoing applications.

The above-referenced PCT international application was published as PCT International Publication No. WO 2023/114990 A1 on Jun. 22, 2023, which publication is incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This technology pertains generally to molecular structure or sequence diagnostic devices and methods and more particularly to a multidimensional spectroscopic tool for simultaneous characterization of the structural and transport properties of a single molecule. The apparatus and system are a combination of a single-molecule tunneling gap with a Raman microscope which allows multi-dimensional information to be extracted about the molecule of interest. The system can be used for analysis, detection, identification, or sensing for a specific chemical or biological species.

### 2. Background

Technological discoveries in the field of nanoscience have allowed for the manipulation and study of matter down to molecular and atomic levels. Nanoscale manipulation and characterization of individual molecules is necessary to understand the intricacies of molecular structure, which governs phenomena such as reaction mechanisms, catalysis, local effective temperatures, surface interactions, and charge transfer and transport.

The recent instrumentation revolution has enabled a shift in physical and analytical chemistry from traditional approaches involving ensemble averaged quantities towards methods capable of investigating the chemical structure and dynamics of individual molecules. These approaches provide important insights into how specific changes in molecular configuration affect catalytic processes, electron transfer and transport properties in molecular and biological systems and have led to the development of novel methods for genomic sequencing.

It has been previously demonstrated that when the components of a biopolymer such as DNA or proteins are passed between two electrodes, there are momentary fluctuations of the current that arise from the change in the tunneling barrier through the subcomponents of the molecular system. This change in the current can be due to intermittent bonding of the molecular species to one or both of the electrodes, or simply due to the change in the tunneling barrier height without requiring any bonding. This current has been demonstrated to be unique to different DNA bases, amino acids, as well as a large variety of other molecules. However, in real systems with large numbers of species, it becomes difficult to disambiguate between species by measuring changes in the current.

It has also been demonstrated that Surface Enhanced Raman Spectroscopy and Tip Enhanced Raman Spectroscopy can be used to identify molecules or even sequence polymers and biopolymers. Although Enhanced Raman spectroscopy (SERS, TERS, etc.) is commonly used to obtain single-molecule Raman spectra, the SERS and TERS procedures are not typically used for sensing or sequencing because of the difficulty of localizing the field and the difficulty of knowing when only a single-molecule is present (to be able to distinguish the molecule of interest).

Techniques in field-enhanced Raman spectroscopy and single-molecule electrical characterization individually have garnered significant attention due to their ability to reliably study the vibrational and electronic structure of individual molecules. However, in general it is very difficult to simultaneously extract multi-dimensional information about the optical, electrical, and mechanical properties of molecules in situ using these approaches due to the complexity of the measurements in any individual domain. This barrier inhibits the ability of exploring the interplay between these properties in single-molecule configurations, and determining how they relate to conformation, isomerism, surface binding configuration, and photon-vibration or electron vibration interactions.

Accordingly, there is a need for new systems, devices and schemes to allow reliable nanoscale manipulation and characterization of the structural, mechanical and electronic transport properties of individual molecules.

### BRIEF SUMMARY

Systems and methods for characterization of the structural and transport properties of a single molecule target are provided that are capable of being used for sensing, spectroscopy, sequencing and molecular identification. The systems generally combine a single-molecule tunneling gap with a Raman spectrometer to allow multi-dimensional information to be extracted from a target molecule of interest for analysis, detection, identification, or sensing for a specific chemical or biological species.

The systems and methods have a Raman module and a single-molecule tunneling gap module that are centered on a substrate with a transverse bore or surface channel located between two opposing electrodes of the break junction. Target molecules are systematically moved relative to the field enhancement region to allow sequence information to be extracted from individual spectra. Data is acquired and processed with a data control and processing module in one embodiment.

It has also been shown that when a single molecule is placed between two electrodes with a bias applied there is a significant enhancement in the Raman signal. In this case a change in the electrical signal occurs when the target molecule is between the two electrodes, and the Raman signal associated with the molecule is greatly enhanced, thus allowing simultaneous electrical and Raman measurements on the same molecular species. This combination of electrical and Raman sensing and identification provides a new opportunity for a multi-dimensional platform for extracting chemical information from analytes of interest, or from the sequence of proteins or nucleic acids or other polymers.

The apparatus has two electrodes that are separated by a sufficiently small distance to allow a current (e.g. tunneling current) to flow while a molecule is placed between them or being translocated between them. The electrodes can be constructed of metals, semiconductors, transparent electrodes, graphene, carbon nanotubes, or 2D electronic materials.

These two electrodes also serve as an optical/plasmonic cavity to enhance the Raman signal to allow a Raman spectrum/chemical fingerprint of the molecule or molecular component between the two electrodes to be obtained. The combination of these two vectors of information improves resolution of the molecular components as they are interrogated by both the electrical signal and the optical signal.

This multi-dimensional spectroscopic platform allows systematic measurement of the structural, mechanical, and electronic transport properties of a single molecule. By performing combined single-molecule, high temporal resolution, Raman spectroscopy with molecular transport measurements in situ, it is possible to extract information concerning the configuration and dynamics of the molecule within the junction under ambient conditions. The combination of time correlated changes in molecular conductance measurements with Raman signatures in both the Stokes and anti-Stokes regions, statistical measurements of changes in the spectra and specific modes when a molecule is bound between two electrodes, and the shifts within modes during these events allows for direct insights into the evolution and modulation of a single-molecule.

Multidimensional information also provides two handles for identification thus greatly improving the reliability of the system. Previous attempts at using tunneling recognition for sensing/sequencing of DNA for instance are very error prone. Adding a Raman signature allows for the identification of when a single base is present in the tunneling gap and the Raman spectrum can provide unambiguous information about what base is present from the spectral fingerprint.

The high-speed nature of the measurements allows for the acquisition of large quantities of data necessary for performing a statistical analysis using machine learning. This analysis acts to verify the integrity of these measurements and to isolate characteristic data sets possessing physically significant features. Thus, this combined system provides a versatile and accessible platform for the continued investigation of chemical structural dynamics at the single-molecule level which could allow for unique insights into phenomena such as field driven chemical reactions, enzymatic processes, and molecular binding and transport processes.

According to one aspect of the technology, a system is provided with recognition tunneling in synergistic combination with Enhanced Raman spectroscopy providing a conductance value and the chemical fingerprint, two vectors for identifying a target molecule.

According to another aspect of the technology, a system is provided with two electrodes in proximity to the pore that are within a tunneling distance of one another to enable the multi-dimensional characterization of the molecules as they pass through the pore.

Another aspect of the technology is to provide a system and apparatus with electrodes on either side of a nanochannel or nano-trench that are within a tunneling distance of one another. Target molecule(s) are translocated through the channel using either electrophoretic or hydrodynamic flow with simultaneous electrical and Raman characterization while passing between the electrodes.

A further aspect is to provide a system and apparatus with a junction with one or two moveable electrodes that are continuously brought into and out of tunneling range to capture the molecules of interest for detection and identification.

Another aspect of the technology is to provide an optical/plasmonic cavity to enhance the Raman signal to obtain a Raman spectrum/chemical fingerprint of the molecule or molecular component between the two electrodes.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is schematic conceptual overview of the system that allows the simultaneous acquisition of tunneling currents and Raman spectra for molecular sensing, detection, identifications or sequencing according to one embodiment of the technology.

FIG. 2 is a schematic view of the system of FIG. 1 with a substrate that has a bore or nanopore disposed between two electrodes and an analyte translocating through the bore for combined single-molecule Raman and conductance analysis.

FIG. 3 is a schematic perspective view of one embodiment of a substrate with a transverse nanopore or bore centered between electrodes of a single molecule junction for orienting a subject target for simultaneous Raman-tunneling gap analysis.

FIG. 4 is a schematic perspective view of an alternative sensing platform with a substrate nanochannel structure centered between electrodes for orienting a subject target for simultaneous Raman-tunneling gap analysis.

FIG. 5 is a detailed perspective schematic view of a MEMS based single molecule junction with opposing electrodes that can be in a fixed position as shown or can be movable towards each other according to one embodiment of the technology.

FIG. 6 is a schematic diagram of a system showing the Raman spectroscopy feature for providing combined single-molecule Raman and conductance measurements with a single molecule junction according to one embodiment of the technology.

FIG. 7 is a functional block diagram of a method for sensing and sequencing using the combined conductance-Raman spectroscopy platform according to one embodiment of the technology.

FIG. 8 is a plot of current response recognition tunneling over time from the single-molecule junction feature of the platform.

FIG. 9 is a plot of a Raman response chemical/vibrational fingerprint of a target of the Raman spectroscopy feature of the platform.

FIG. 10 is a grid of aggregated data from hundreds of spectra and conductance values for 6 different amino acids that demonstrate that there is a combination of unique spectral and conductance features that can be used to allow identification (detection) of individual amino acids.

FIG. 11 is a plot of predicted versus actual results using machine learning techniques to identify specific amino acids from the combined Raman/Conductance data with accuracies ranging from 60-83%.

FIG. 12 is a plot of a Raman spectra comparison between the MEMS-BJ response for cysteine (top), the nanopore device (middle, obtained at the pore), and the PDMS background (bottom).

### DETAILED DESCRIPTION

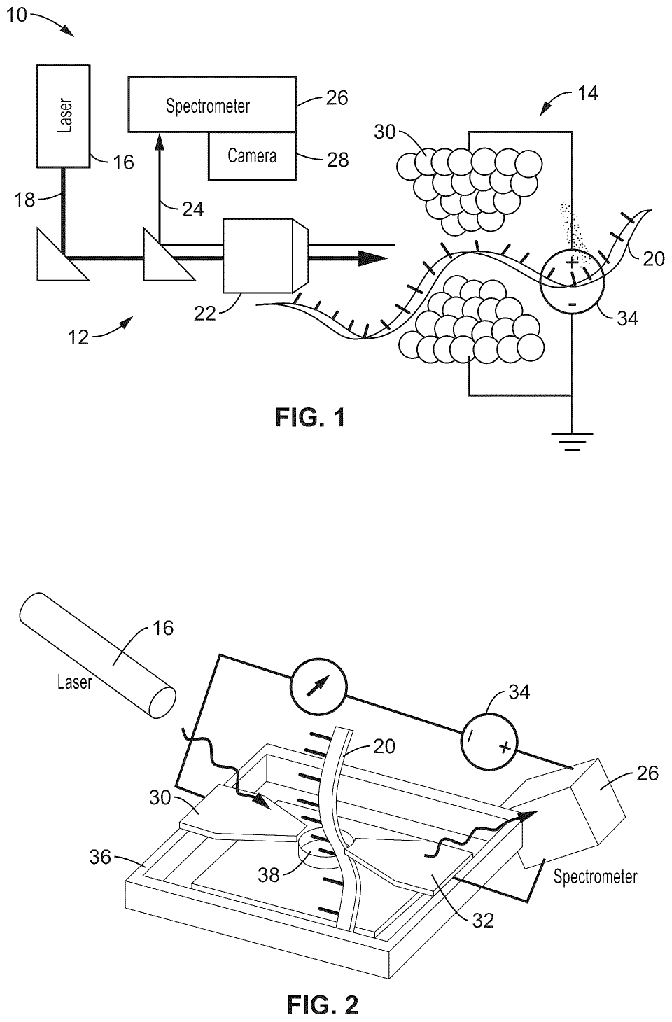

Referring more specifically to the drawings, for illustrative purposes, systems and methods for the simultaneous characterization of the electrical, optical, and mechanical properties of a single molecule using combined single-molecule Raman and conductance measurements to extract structural information about a molecule are generally shown. Several embodiments of the technology are described generally in FIG. 1 to FIG. 12 to illustrate the characteristics and functionality of the devices, systems, materials and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

Turning now to FIG. 1, the general structure of one apparatus and system 10 for combined single-molecule Raman and conductance analysis is shown schematically. The apparatus of system 10 is useful for chemical sensing and analysis, for instance, of DNA/RNA base recognition/sequencing, amino acid/peptide identification, protein sequencing, chemical detection for security or food processing, etc.

The apparatus and system provides multi-vector sensing capabilities for chemical and biological species that will enable direct detection and identification of chemical species, or in the case of long-sequence polymers (proteins, DNA, RNA, peptides) allow sequencing. This is accomplished by combining the recognition tunneling capabilities demonstrated in single-molecule electronic systems with single-molecule Raman spectroscopy, which provides a chemical fingerprint of a molecular species and a sensing substrate with a transverse bore or channel that orients the target. In this case, the current between the two electrodes and the Raman signal provide complementary information about the molecular species to improve sensing, sequencing, and identification capabilities over each process individually.

Raman enhancement between two nanostructured electrodes in combination with direct charge transport measurements allows simultaneous characterization of the electrical, optical, and mechanical properties of a single molecule. This multi-dimensional information yields repeatable, self-consistent, verification of single-molecule resolution, and allows for detailed analysis of structural and configurational changes of the molecule in situ. Experimental results may be supported by a machine-learning based or conventional statistical analysis of the spectral information and calculations to provide insight into the correlation between structural changes in a single-molecule and its charge-transport properties.

As shown schematically in FIG. 1 and FIG. 2, the apparatus of system 10 has a Raman spectroscopy module 12 and a single molecule junction module 14. The Raman module generally comprises a Raman excitation source such as a laser 16 directing a beam 18 to a target 20 through suitable optics 22. Raman spectra 24 is returned through optics 22 to the spectrometer 26 and recorded by camera 28.

The single molecule break junction module 14 of the apparatus and system 10 includes two opposing nanostructured electrodes with a controllable distance between them in a single-molecule break junction (SMBJ) configuration are used to simultaneously create an optical enhancement cavity and a single-molecule electrical junction. The opposing first electrode tip 30 and second electrode tip 32 may be stationary or movable in relation to each other and are electrically coupled to an electrical bias and conductance monitor 34. The target molecules of interest 20 are localized between the two electrodes 30, 32.

Electrode spacing for tunneling is preferably on the order of 1 nm to 2 nm, depending on the molecular size, the coating of binding molecules, etc. There is also the possibility that when the molecule binds to the charge transport mechanism there will be hopping or resonant tunneling rather than “normal” tunneling, which can allow currents to be measured up to slightly larger distances (e.g. still focused at <10 nm).

The electrodes are preferably made from noble metals, indium tin oxide (ITO), fluorine tine oxide (FTO), graphene or other conductive materials and have a nano scaled size. The electrodes 30, 32 may also be coated with other molecules to help improve binding with target molecules, dwell time and current and Raman signals. The molecules can also be thiolated to bind to noble metals during translocation. From a current standpoint, the addition of molecules to the tip of the electrodes for temporary binding, likely via hydrogen binding, can improve the current signal, but also improve the ability to obtain the Raman spectra because of the enhanced polarizability of the molecule.

In one embodiment, one or both of the electrodes are coated in a non-conductive material except at the tip. In another embodiment, one or both electrodes have atomic/nanoscale roughness that enhances the field.

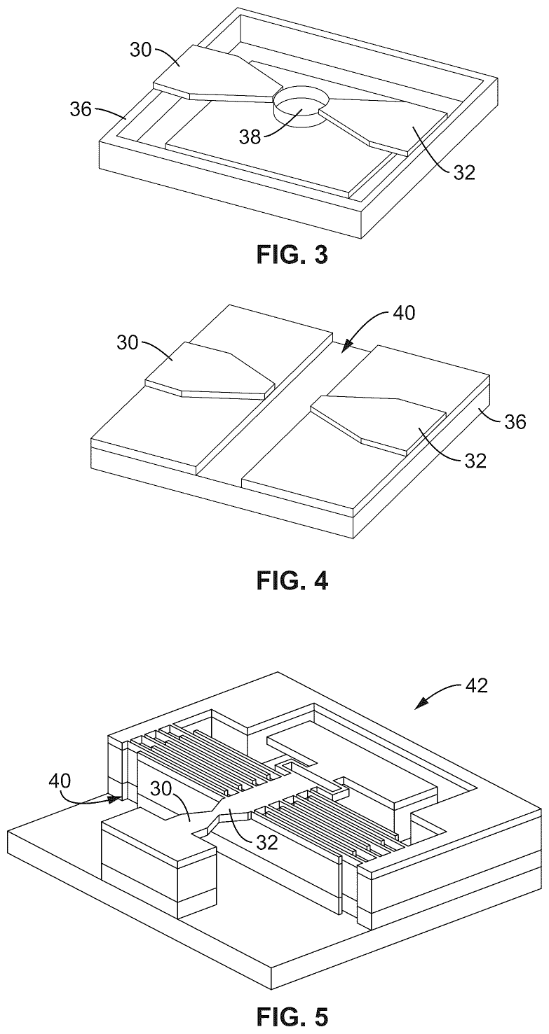

The two modules 12,14 of system 10 are centered over a substrate 36 to perform the combined single-molecule Raman and conductance analysis. In the embodiment shown in FIG. 2 and FIG. 3, the substrate 36 has a transverse nanopore or bore 38 that is preferably centered between the electrodes 30, 32. Target molecules of interest 20 are translocated through the nanopore 38 for sequential analysis.

In another embodiment of the substrate 36, shown in FIG. 4, the substrate 36 has a channel 40 between the electrodes 30, 32 that allows a flow of material between the electrodes. For example, a MEMS based junction 42 with one stationary electrode 30 and one opposing moveable electrode 32 and channel 40 is shown in FIG. 5. The distance between the two nanostructured electrodes 30, 32 is controllable in a single-molecule break junction configuration that can simultaneously create an optical enhancement cavity and a single molecule electrical junction.

The functional significance of the nanopore 38 or channel 40 structures is that it allows for control of the translocation of the target 20 in a linear fashion while constraining the target molecule into a single dimension, in a planar substrate there is no way to identify which sub-component of the molecule may be between the two electrodes. Therefore, having some constriction may be desirable. Pore 38 and channel 40 have sizes that are preferably <10 nm in dimension and about 2 nm is particularly preferred.

Translocation of targets 20 through the nanopore 38 or channel 40 can be accomplished in several ways including through the use of electrophoresis or electrochemical forces or hydrodynamic forces. The target may also be coupled to a nanoparticle at one end and drawn through the pore by magnetic field control or optical tweezers or traps.

In one embodiment, sequencing through a nanopore system is conducted with the following steps:
1) The electrodes could be coated with a molecule capable of temporary binding (e.g. van der Waals or hydrogen bonding) to the molecular sub-components during translocation;
2) A nanopore system would be placed between two reservoirs;
3) Single-Stranded DNA, RNA, or denatured (unfolded) protein or peptide placed in one chamber, for example. Note: Denaturing can be done with heat or certain chemical reagents e.g. urea or pH control;
4) An electric field can be used to translocate the entities of interest through the pore structure; In another embodiment, magnetic or optical trapping can also be used here if one end the molecule is modified with an appropriate label for each technique. Hydrodynamic pressures can also be used for label-free control);
5) As the molecule translocates, the bases, amino acids or molecules etc. can temporarily bind to the electrodes to allow recognition tunneling and simultaneously obtain the Raman spectra; and
6) Multiple translocations can be done to provide redundant testing sequencing for multiple reads and improve accuracy. The channel structure would proceed similarly.

The previously recited structures and systems 10 with a nanopore 38 or channel 40 structures permits the sequential or incremental translocation of a target through a sensing point and preferably includes a plasmonic nanocavity 44 to improve the Raman enhancement. This permits simultaneous Raman and electrical conductance measurements of molecules or sub-components of molecules.

Tip-Enhanced Raman Spectroscopy (TERS) has been demonstrated to allow sub-molecular resolution of molecular vibrations by taking advantage of the large field enhancement between a metallic tip that operates as a nanoscale antenna and a conductive surface. It has also been demonstrated that plasmonic cavities can exist between two such nano-antennas. These cavities can support gap surface plasmons which results in extremely large and extremely localized enhancement of an incident electromagnetic field. These conditions enable the Raman spectra of a single molecule to be extracted from extremely small volumes such as found at the junction formed between a molecule and two metal contacts, and that this enhancement changes even when a molecule binds and unbinds within a junction. When a junction is formed by a molecule bridging the gap between the nano-antennas, the electromagnetic field enhancement needed to resolve the Raman scattering from a single molecule is highly localized and drops off rapidly when the junction breaks.

Plasmonic nanocavities 44 improve the Raman enhancement the smaller the size of the gap, and small perturbations in structures (such as metal adatoms) can create hotspots with huge enhancement factors. These are sometimes referred to as picocavites.

To create a significantly improved field, an electrode separation, on the order of 1 nm to 2 nm, provides a huge electromagnetic enhancement factor when the molecule is bound between the two electrodes, which allows the spectrum from that molecule to be separated from the background Raman spectrum. This also correlates with the exact moments that the recognition tunneling events occur.

It can be seen that the break junction electrode structures generally form the basis of the plasmonic nanocavity 44 where the laser 16 can create plasmons in the electrodes that enhances the field and Raman scattering and allows simultaneous recognition tunneling and Raman spectroscopy for identifying molecular substituents.

To improve the current response and Raman response of the apparatus, in one embodiment, the frequency of the applied laser 16 is matched to the plasmon frequency of the electrode materials to enhance the Raman scattering.

In another embodiment, a separate plasmonic structure is placed within the junction area to further enhance the plasmonic/nanocavity effect. For example, nanoparticles or a metal layer can be placed on the substrate surfaces surrounding the nanochannel or nanopore within or proximal to the break junction.

In another embodiment, the metal electrodes 30, 32 of the junction have surfaces with atomic/nanoscale roughness that may also enhance the fields and the plasmonic/nanocavity effect.

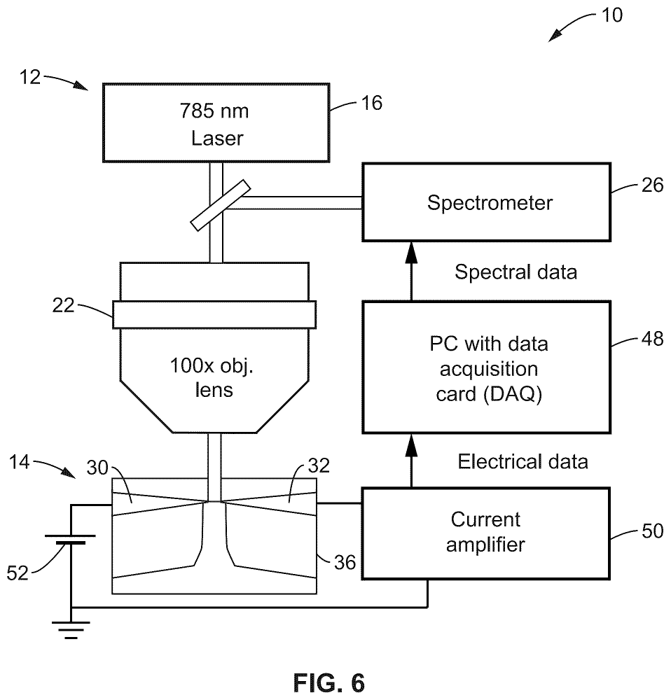

The system components, including the spectrometer 26, electrode 30, 32 movement, laser 16 actuation and frequency, may also be controlled by a computer and programming 48 as illustrated generally in FIG. 6. The controller 48 controls the components and functions of the Raman module 12 and the break junction module 14. For example, the controller 48 can control the frequency of the laser 16 as well as control whether the laser beam is continuous or chopped. The controller 48 can also control the current amplifier 50 electrode bias 52, detector 34 as well as target translocation mechanisms for movement through the substrate 36. The system controller 48 may also record and process the data acquired from the Raman components as well as the conductance and other data acquired from the electrodes 30, 32. The system controller may also have programming that evaluates the acquired data, for example, by comparing the acquired spectra with a database or using Machine Learning algorithms to recognize or identify the molecules that bind in the junction in real time. The system controller may also display results and analysis on a display.

By applying a bias 52 between the two electrodes 30, 32 while simultaneously measuring the Raman spectra from the junction with the spectrometer 26, it is possible to perform combined, single-molecule junction enhanced Raman and conductance spectroscopies (JERCS) with high temporal resolution. This multi-dimensional spectroscopic platform 10 allows simultaneous measurement of the structural, mechanical, and electronic transport properties of a single molecule.

Unique to the setup is its operation in the absence of feedback allowing single-molecule junctions to be sustained on time scales of several hundred milliseconds. Additionally, notably short integration times (e.g. 22 ms) may be used when acquiring spectral data. These features allow the observation of intricate changes of the molecule over the course of the lifetime of the junction.

By correlating measurements of the electron transport and enhanced Raman scattering of a single molecule 20 bound between two nanostructured electrodes 30, 32, mutually verifiable evidence of single-molecule Raman scattering with millisecond temporal resolution and microsecond resolution with the use of high-speed cameras 28 can be obtained. This allows for the simultaneous extraction of a chemical fingerprint from the molecule target 20 along with information about its configuration, and the strain applied to individual bonds, and to see how these properties relate to the charge transport properties within the molecular junction. Combined, this multi-dimensional information provides significant insights into single-molecule structural dynamics.

The high-speed nature of the measurements allows for the acquisition of large quantities of data necessary for performing a statistical analysis using machine learning. This analysis acts to verify the integrity of these measurements and to isolate characteristic data sets possessing physically significant features. Thus, this combined JERCS system provides a versatile and accessible platform for the continued investigation of chemical structural dynamics, chemical identification, and sequencing at the single/sub-molecular level which could allow for unique insights into phenomena such as field driven chemical reactions, enzymatic processes, and molecular binding and transport processes.

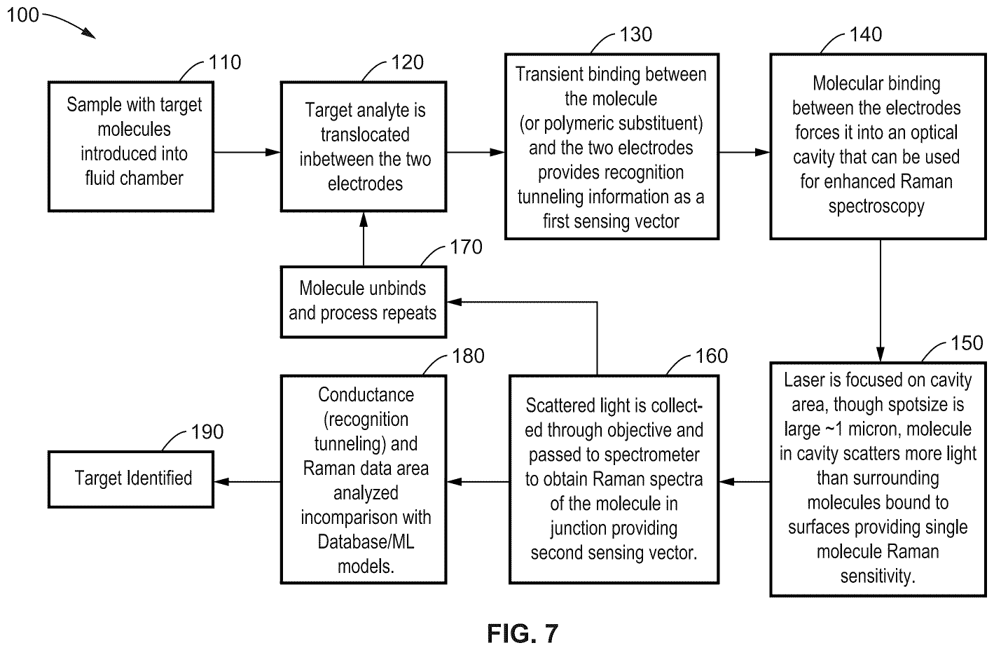

Turning now to FIG. 7, a block diagram of a method 100 for sensing and sequencing with combined conductance and Raman spectroscopy platform is generally shown. At block 110, the sample target is introduced into the system 10 with either a pore or a channel with two nanoscopic electrodes transverse to the pore (or channel) as shown in FIG. 2. The target is then translocated between the two electrodes at block 120. A force can be applied to force a molecule (or a polymer, biopolymer, etc.) through the pore (channel). This can be done by diffusion, (di)-electrophoresis, magnetic fields, pressure, hydrodynamic flows, etc.

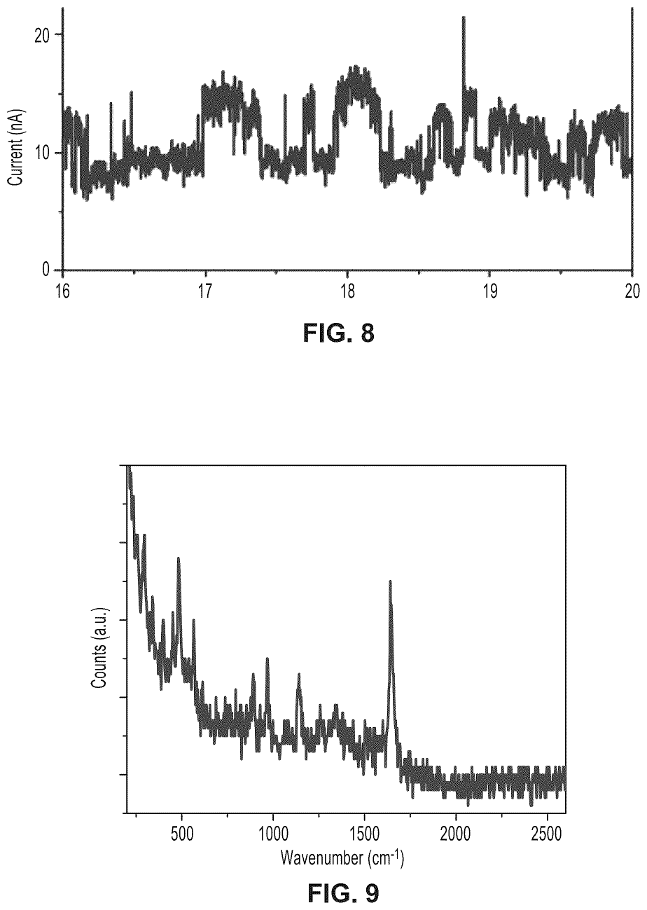

As the target moves through the pore (channel) it may momentarily bind between the two electrodes providing a current (conductance) signal identifying the presence of a single-molecule at block 130. This is commonly referred to as “recognition tunneling” as the conductance is determined by the chemical nature of the molecule. However, conductance dispersion may be very large (˜ 1 order of magnitude), making it difficult to discriminate between many potential molecular species (e.g. different DNA bases). Current response of recognition tunneling is illustrated in FIG. 8.

The small separation between the two electrodes also creates an optical (or plasmonic) cavity which can enhance the electric field from a laser applied to the junction. This enhanced electric field can allow simultaneous enhanced Raman spectroscopy of whatever is between the electrodes at block 140 of FIG. 7.

At block 150, single Raman spectra is obtained. Typically, a laser is focused on the cavity between the electrodes and scattered light collected and detected at block 160. The scattered light is collected through the objective and directed to the spectrometer for detection and analysis of the Raman spectra. The Raman spectra provides a chemical/vibrational fingerprint of the molecule of interest as illustrated in FIG. 9.

Thus, the combined system provides strict localization of the enhancement effect, direct information about when a single-molecule is present (through the recognition tunneling), a strong enhancement of the molecule in the junction allowing single-molecule spectra to be obtained, and two vectors for identifying the molecule: the conductance value and the chemical fingerprint. It is the synergy between these effects that provides a unique sensing motif.

After break junction/recognition tunneling and Raman data acquisition at block 140 and block 160, the target molecule unbinds from the electrodes and the process repeats at block 170 of binding a new target with the electrodes. Because the electrode binding is transient it can allow throughput sensing of many molecules or sequential identification of molecular substituents along a biopolymer, for example.

The simultaneously acquired Raman data and recognition tunneling data over time are analyzed at block 180. The data from single-molecule junction enhanced Raman and conductance spectroscopies can be compared with a database of prior results or compared with one or more models at block 180. Such comparisons assist in the identification of the target sequence or molecule at block 190. Sequencing is also possible at block 190 by combining the recognition tunneling capabilities demonstrated in single-molecule electronic systems with single-molecule Raman spectroscopy, which provides a chemical fingerprint of a molecular species.

Accordingly, a multidimensional spectroscopic tool is provided for simultaneous characterization of the structural and transport properties of a single molecule. By correlating measurements of the electron transport and enhanced Raman scattering of a single molecule bound between two nanostructured electrodes, we provide mutually verifiable evidence of single-molecule Raman scattering with millisecond temporal resolution. This allows simultaneous extraction of a chemical fingerprint from the molecule under study along with information about its configuration, and the strain applied to individual bonds, and how these properties relate to the charge transport properties within the molecular junction. Combined, this multi-dimensional information provides significant insights into single-molecule structural dynamics and chemical and structural information.

The technology described herein may be better understood with reference to the accompanying examples, which are intended for purposes of illustration only and should not be construed as in any sense limiting the scope of the technology described herein as defined in the claims appended hereto.

### EXAMPLE 1

In order to demonstrate the functionality of the system and methods, one embodiment of the combined conductance/Raman spectroscopy platform device was constructed and basic methods tested. Device electrodes were fabricated with tips modeled with a radius of 90 nm on a flat gold substrate. The selected target was Biphenyl-4,4′-dithiol (BPDT).

The BPDT molecule was brought in tunneling distance with both tips of the electrodes and then illuminated by a Raman excitation source. The single-molecule junctions were sustained on time scales of several hundred milliseconds. A bias was applied between the two electrodes while simultaneously measuring the Raman spectra from the junction and it was possible to perform combined, single-molecule junction enhanced Raman and conductance spectroscopy with high temporal resolution.

Once the system stabilized, the feedback was turned off, and stochastic jumps in the current appeared over time which represented a molecule intermittently binding between the two electrodes. These events were observed to be strongly correlated with a large increase in the Raman intensity in both the Stokes (positive) and anti-Stokes (negative) regions of the obtained spectra.

A decrease in Raman intensity was also observed when the molecular junction broke down as evidenced by a sudden decrease in the junction conductance. Because this field enhancement falls off rapidly with the distance from the center of the cavity, nearby molecules will have a minimal contribution to the Raman signal when compared to a molecule that is bound to both electrodes and at the maximum of the field enhancement.

The intense electromagnetic enhancement observed within the cavity at the point of junction formation combined with the potential chemical enhancement from the Au-thiol bond formation on both sides of the molecule draw an important distinction between junction enhancement and more traditional TERS.

The combination of a strong correlation between the intensity of anti-Stokes scattering with the formation of a junction suggested that the anti-Stokes scattered photons are obtained when the molecule is transporting electrons. Furthermore, the ratio of anti-Stokes to Stokes peak intensity showed a clear dependence on the applied external bias when the optical power was held constant. This suggested that as the bias increases, electron scattering through the junction increases and the resultant enhancement is not induced by optical pumping of the target molecule.

### EXAMPLE 2

To determine if specific modes or spectral features could be used to clearly identify when a single-molecule spectra/junction is obtained, an unsupervised machine learning approach was used to obtain a statistically significant “fingerprint” for junction formation. The observed clustering 10955 BPDT Raman spectra in the 1200 to 1700 cm⁻¹ region were collected during conduction measurements. The corresponding conductance data is was used to verify that the spectral features correspond when a single-molecule junction is formed between the two electrodes.

Intensity plots of the spectral data in this region were grouped through k-means clustering into 2 clusters to separate single-molecule events and background spectra. The two clusters showed drastic differences in their spectral features. Spectral lines in the first cluster (denoted as “On Cluster”) had notably higher intensities and greater variation than the other cluster (“Off Cluster”) which was significantly lower in intensity. A histogram of the conductance corresponding to the measured spectrum in each cluster showed a sharp distribution centered at the characteristic conductance of BPDT for the On Cluster while the histogram for the Off Cluster showed a distribution with a much wider breadth centered at a lower conductance value near the setpoint current for the measurements.

The outcome of this analysis implied two important capabilities for single molecule system measurements. First, it allows the identification of spectral features that are representative of the formation of a single-molecule junction to analyze changes in molecular configuration vs. idealized structures. And second, it enables the processing of a large amount of recorded data to find long lasting coupling events with correlated spectral and conductance features. The information contained in these measurements allows the analysis of dynamic processes that occur within the single molecule junction and provides an opportunity to understand the changing mechanics of these systems and how structure relates directly to the properties of single-molecule systems such as electron transport.

To determine specific spectral features, the average spectra of each cluster and density functional theory (DFT) calculations of the molecule in-contact and out-of-contact were used as a basis for comparison. Several prominent features were present in the On Cluster data that were not apparent in the Off Cluster data such as the v(6a) mode at ca. 350 cm⁻¹, and the v(17b) mode at ca. 800 cm⁻¹. However, the vibrational mode related to the v(8a) ring vibration is the most prominent and is clear in both clusters. By fitting the calculated Raman shift of the v(8a) mode and conductance from our model system to the experimental data it was possible to visualize the dynamics of the junction throughout the coupling event.

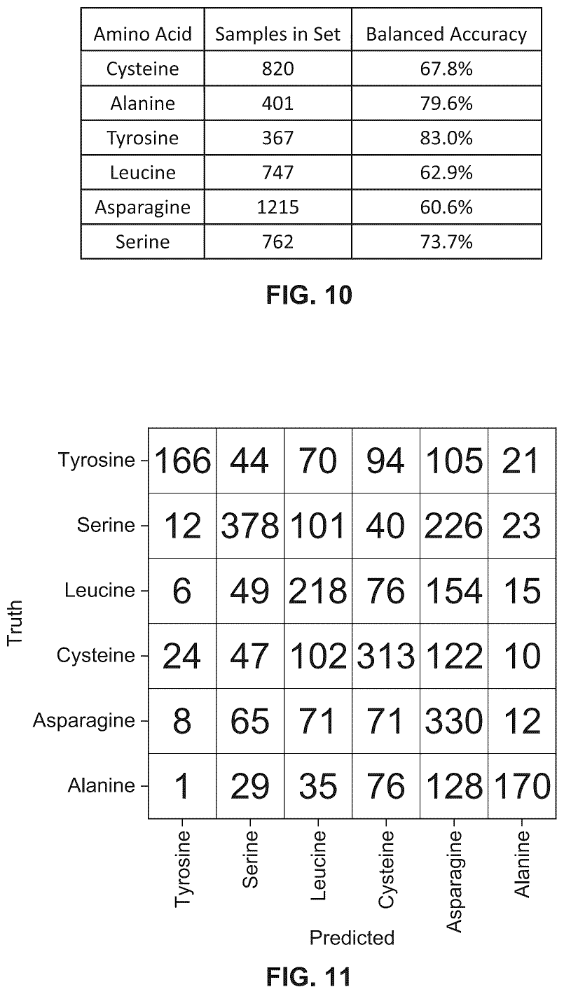

Aggregation of data from hundreds of spectra and conductance values for 6 different amino acids demonstrated that there is a combination of unique spectral and conductance features that can be used to allow identification or detection of individual amino acids, as shown in FIG. 10.

Using machine learning techniques it is possible to identify specific amino acids from the combined Raman/Conductance data with accuracies ranging from 60-83% as illustrated in FIG. 11. This accuracy may be further improved as more data is acquired and more sophisticated data processing procedures are implemented.

### EXAMPLE 3

To further evaluate the functionality of the devices, a nanopore structure was constructed to demonstrate sensing and movement with cysteine as a target. Raman Spectroscopy on a nanopore structure with cysteine applied was conducted and an optical micrograph of nanopore after electrochemical electrode deposition was taken. The Raman map of gap region after flowing cysteine through the gap region showed clear enhancement at surface edges and within the opening/gap.

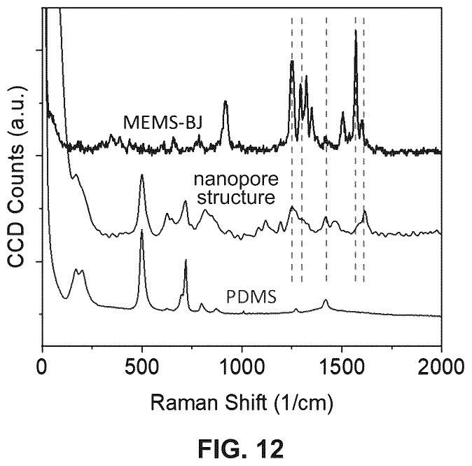

A plot of the Raman responses at different locations in the structure for comparison is shown in FIG. 12. The Raman spectra comparison between the MEMS-break junction response for cysteine (top), for the nanopore device (middle, obtained at the pore), and the PDMS background (bottom). The vertical dashed lines on FIG. 12 indicate several features present in both the MEMS-BJ and nanopore spectra. The gap region has features of both the target of interest (cysteine) and the PDMS background, suggesting that target Raman spectra can be obtained even in the presence of background scattering from the surrounding structures.

From the description herein, it will be appreciated that the present disclosure encompasses multiple embodiments which include, but are not limited to, the following:

An apparatus for chemical identification, the apparatus comprising: (a) a substrate with a transverse bore through the substrate opening to an upper surface: (b) a Raman spectrometer having an excitation source, a sampling apparatus, and a detector configured to interrogate the bore; (c) two or more opposing nanoscale electrodes with electrodes positioned on either side of the bore at selected distances; and (d) a data acquisition unit configured for receiving spectral data from the Raman spectrometer detector and for receiving electrical data from the electrodes; (e) wherein simultaneous Raman spectroscopy and electrical current measurements are made.

An apparatus for chemical identification, the apparatus comprising: (a) a substrate with a channel; (b) a Raman spectrometer having an excitation source, a sampling apparatus, and a detector configured to interrogate a section of the channel; (c) two or more opposing nanoscale electrodes with electrodes positioned on various sides of the section of substrate channel at selected distances; and (d) a data acquisition unit configured for receiving spectral data from the Raman spectrometer detector and for receiving electrical data from the electrodes; (e) wherein simultaneous Raman spectroscopy and electrical current measurements are made.

The apparatus of any preceding or following implementation, further comprising: a means for translocating a target through the substrate bore and proximal to the electrodes for analysis.

The apparatus of any preceding or following implementation, further comprising: a means for translocating a target along the channel and proximal to the electrodes for analysis.

The apparatus of any preceding or following implementation, wherein the means for translocation is a process selected from the group consisting of electrophoresis, electrochemical forces, optoelectric, magnetic, pressure, and capillary processes.

The apparatus of any preceding or following implementation, wherein the excitation source is a laser selected from the group of a variable frequency laser, a continuous wave laser, a pulsed laser and a chopped beam laser.

The apparatus of any preceding or following implementation, further comprising: (a) a MEMS-based actuator coupled to the electrodes; (b) a first conductive needle electrode coupled to the MEMS-based actuator; and (c) a second conductive needle electrode; (d) wherein the first conductive needle electrode is configured to move relative to the second conductive needle electrode; (e) wherein the first conductive needle electrode is moveable in positional relation to the second electrode by the MEMS-based actuator; and (f) wherein, optionally, the first conductive needle electrode and second conductive needle electrode are formed from breaking a single conductive filament.

The apparatus of any preceding or following implementation, wherein the second conductive needle electrode further comprises: (a) a second MEMS-based actuator; and (b) the second conductive needle electrode coupled to the second MEMS-based actuator; (c) wherein the second conductive needle electrode is moveable in positional relation to the first conductive needle electrode by the second MEMS-based actuator.

The apparatus of any preceding or following implementation, wherein the first or second conductive needle electrode has a tip that is coated with a conductive metal selected from the group of gold, platinum, rhodium, ruthenium, other transition metals and titanium and alloys.

The apparatus of any preceding or following implementation, wherein the opposing electrodes are made of a material selected from the group of materials consisting of Noble metals, indium tin oxide, fluorine tin oxide, graphene, semi-conductive materials, carbon nanotubes, conductive polymers, conductive metal oxides, amorphous materials, and other electronic 2D materials.

The apparatus of any preceding or following implementation, wherein the electrodes of the single-molecule break junction further comprises a surface coating of molecules to improve target molecule binding during translocation, improve dwell time and current and Raman signals.

A method for chemical identification, with a combined Raman/electrode system, the method comprising: (a) translocating a target molecule incrementally along a channel in a substrate and between electrodes forming a junction for analysis; (b) performing electrical current transport measurements on the target molecule within the junction; (c) simultaneously performing Raman spectroscopy on the target molecule or sub-component of the molecule while it is located in the junction; (d) recording current and spectral measurements; and (e) analyzing the current and spectral measurements.

A method for chemical identification, with a combined Raman/electrode system, the method comprising: (a) translocating a target molecule incrementally through a transverse bore within a substrate positioned between electrodes forming a junction for analysis; (b) performing current measurements on the target molecule within the junction; (c) simultaneously performing Raman spectroscopy on the target molecule while it is located in the junction; (d) recording current and spectral measurements; and (e) analyzing the current and spectral measurements.

The method of claim any preceding or following implementation, further comprising controlling the translocation, current measurements, spectroscopy measurements and recording with a controller.

The method of claim any preceding or following implementation, further comprising treating target molecules with thiols or other chemical linkers to improve binding to electrodes during translocation.

The method of any preceding or following implementation, further comprising continuously moving a pair of moveable electrodes into and out of contact to capture the molecules of interest for detection and identification.

The method of any preceding or following implementation, further comprising controlling the movement of each moveable electrode; controlling a bias applied to the electrodes; and controlling timing of Raman spectroscopy acquisitions.

The method of any preceding or following implementation, further comprising controlling an electrophoretic or hydrodynamic flow translocating a target through a channel or a substrate bore and through the junction for analysis.

The method of any preceding or following implementation, further comprising computing chemical information of the target from recorded current and spectral measurements.

An apparatus comprising: a) a Raman spectroscope; b) a single-molecule junction electronic current sensor; c) a mechanism for feeding a biopolymer molecule through a junction; and d) a computer to record and store sequence information.

An apparatus, comprising: (a) a planar substrate with a linear channel in an upper surface: (b) a Raman spectrometer having an excitation source, a sampling apparatus, and a detector configured to interrogate a section of the channel; (c) a single-molecule break junction of opposing nanoscale electrodes with an electrode positioned on either side of the section of substrate channel at a selected distance; and (d) a data acquisition unit configured for receiving spectral data from the Raman spectrometer detector and for receiving conductance data from the break junction; (e) wherein simultaneous single-molecule Raman spectroscopy and electrical molecular current/conductance measurements are made in real time.

An apparatus, comprising: (a) a planar substrate with a transverse bore through the substrate opening to an upper surface: (b) a Raman spectrometer having an excitation source, a sampling apparatus, and a detector configured to interrogate the bore; (c) a single-molecule break junction of opposing nanoscale electrodes with an electrode positioned on either side of the bore at a selected distance; and (d) a data acquisition unit configured for receiving spectral data from the Raman spectrometer detector and for receiving conductance data from the break junction; (e) wherein simultaneous single-molecule Raman spectroscopy and molecular conductance measurements are made in real time.

A method for chemical identification, sequencing and sensing with a combined Raman/single molecule break junction system, the method comprising: (a) translocating a target molecule incrementally along a channel in a substrate and between electrodes forming a break junction for analysis; (b) performing electrical transport measurements on the target molecule within the break junction; (c) simultaneously performing Raman spectroscopy on the target molecule or sub-component of the molecule while it is located in the break junction; (d) recording conductance and spectral measurements; and (e) analyzing the conductance and spectral measurements.

A method for chemical identification, sequencing and sensing with a combined Raman/single molecule break junction system, the method comprising: (a) translocating a target molecule incrementally through a transverse bore within a substrate positioned between electrodes forming a break junction for analysis; (b) performing conduction current measurements on the target molecule within the break junction; (c) simultaneously performing Raman spectroscopy on the target molecule while it is located in the break junction; (d) recording conductance and spectral measurements; and (e) analyzing the conductance and spectral measurements.

The apparatus or method of any preceding or following implementation wherein, the electrodes are coated in a non-conductive material except at the tip.

The apparatus or method of any preceding or following implementation wherein, the electrode tips are coated with molecules for temporary binding, (e.g. hydrogen binding) configured to improve the current signal as well as improve the ability to obtain the Raman spectra because of the enhanced polarizability of the molecule.

The apparatus or method of any preceding or following implementation, wherein the electrodes have atomic/nanoscale roughness that enhances the field.

The apparatus or method of any preceding or following implementation, further comprising: a plasmonic structure within the junction area to further enhance the plasmonic/nanocavity effect.

The apparatus of any preceding or following implementation, wherein the additional plasmonic structure comprises: a layer of metal nanoparticles or metal film on the substrate beneath the electrodes of the junction.

The method of any preceding or following implementation, further comprising: matching the laser frequency to a plasmon frequency of the electrode materials, wherein the laser creates plasmons in the electrodes and enhances the field and Raman scattering.

As used herein, term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these group elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system or method.

As used herein, the term “set” refers to a collection of one or more objects. Thus, for example, a set of objects can include a single object or multiple objects.

Relational terms such as first and second, top and bottom, and the like may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3º, less than or equal to ±2°, less than or equal to ±1°, less than or equal to ±0.5°, less than or equal to ±0.1°, or less than or equal to +0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential features or elements of the technology describes herein or any or all the claims.

In addition, in the foregoing disclosure various features may grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after that application is filed. Accordingly the reader should consult the application as filed for the original content of the disclosure. Any deletion of content of the disclosure should not be construed as a disclaimer, forfeiture or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. An apparatus, comprising:
(a) a planar substrate with a transverse bore through said substrate opening to an upper surface:
(b) a Raman spectrometer having an excitation source, a sampling apparatus, and a detector configured to interrogate said bore;
(c) a single-molecule break junction of opposing nanoscale electrodes with an electrode positioned on either side of said bore at a selected distance; and
(d) a data acquisition unit configured for receiving spectral data from the Raman spectrometer detector and for receiving conductance data from said break junction;
(e) wherein simultaneous single-molecule Raman spectroscopy and molecular conductance measurements are acquired in real time.

2. An apparatus, comprising:
(a) a planar substrate with a linear channel in an upper surface:
(b) a Raman spectrometer having an excitation source, a sampling apparatus, and a detector configured to interrogate a section of said channel;
(c) a single-molecule break junction of opposing nanoscale electrodes with an electrode positioned on either side of said section of substrate channel at a selected distance; and
(d) a data acquisition unit configured for receiving spectral data from the Raman spectrometer detector and for receiving conductance data from said break junction;
(e) wherein simultaneous single-molecule Raman spectroscopy and electrical molecular current/conductance measurements are acquired in real time.

3. The apparatus of claim 1 or claim 2, further comprising:
a plasmonic structure beneath the break junction electrodes to enhance Raman signals.

4. The apparatus of claim 3, wherein said additional plasmonic structure comprises:
a layer of metal nanoparticles or metal film on the substrate beneath the electrodes of the junction.

5. The apparatus of claim 1 or claim 2, further comprising:
a translocator element configured to translocate a target through said substrate bore or along said channel and between said electrodes for analysis, said translocator element using a process selected from the group consisting of electrophoresis, electrochemical forces, optoelectric, magnetic, pressure, and capillary processes.

6. The apparatus of claim 1 or claim 2, wherein said electrodes have atomic or nanoscale roughness formed on outer surfaces of the electrodes.

7. The apparatus or method of claim 1 or claim 2 wherein, said electrodes are coated with a non-conductive material except at the tip.

8. The apparatus of claim 1 or claim 2, further comprising:
(a) a MEMS-based actuator coupled to said electrodes;
(b) a first conductive needle electrode coupled to the MEMS-based actuator; and
(c) a second conductive needle electrode;
(d) wherein the first conductive needle electrode is configured to move relative to the second conductive needle electrode;
(e) wherein the first conductive needle electrode is moveable in positional relation to the second electrode by the MEMS-based actuator; and
(f) wherein, optionally, said first conductive needle electrode and second conductive needle electrode are formed from breaking a single conductive filament.

9. The apparatus of claim 8, wherein said second conductive needle electrode further comprises:
(a) a second MEMS-based actuator; and
(b) said second conductive needle electrode coupled to the second MEMS-based actuator;
(c) wherein the second conductive needle electrode is moveable in positional relation to the first conductive needle electrode by the second MEMS-based actuator.

10. The apparatus of claim 8, wherein said first or second conductive needle electrode has a tip that is coated with a conductive metal selected from the group of gold, platinum and titanium.

11. The apparatus of claim 1 or claim 2, wherein said opposing electrodes are made of a material selected from the group of materials consisting of Noble metals, indium tin oxide, fluorine tin oxide, graphene, semi-conductive materials, carbon nanotubes and other electronic 2D materials.

12. The apparatus of claim 1 or claim 2, wherein said electrodes of the single-molecule break junction further comprises:
a surface coating of molecules to improve target molecule binding during translocation, improve dwell time and current and Raman signals.

13. The apparatus of claim 1 or claim 2, wherein said excitation source is selected from the group consisting of a monochromated light source, a variable frequency laser, a continuous wave laser, a pulsed laser and a chopped beam laser.

14. The apparatus of claim 1 or claim 2, further comprising:
a controller configured to control the Raman spectrometer, single-molecule break junction and data acquisition unit.

15. A method for chemical identification, the method comprising:
(a) translocating a target molecule incrementally through a transverse bore or a channel within a substrate positioned between electrodes forming a junction for analysis;
(b) performing current measurements on the target molecule within the junction;
(c) simultaneously performing Raman spectroscopy on the target molecule while it is located in the junction;
(d) recording current and spectral measurements; and
(e) analyzing the current and spectral measurements.

16. The method of claim 15, further comprising:
forming a plasmon cavity beneath the junction electrodes; and
matching a laser frequency to a plasmon frequency of the electrode materials, wherein said laser creates plasmons in the electrodes and enhances the field and Raman scattering.

17. The method of claim 15, further comprising:
controlling the translocation, current measurements, spectroscopy measurements and recording with a controller.

18. The method of claim 17, further comprising:
controlling an electrophoretic or hydrodynamic flow translocating a target through a channel or a substrate bore and through said junction for analysis.

19. The method of claim 17, further comprising:
controlling the movement of each moveable electrode;
controlling a bias applied to the electrodes; and
controlling timing of Raman spectroscopy acquisitions.

20. The method of claim 17, further comprising:
treating target molecules with thiols or other chemical linkers to improve binding to electrodes during translocation.
